(19) United States
(12) Patent Application Publication
(10) Pub. No.: US 2009/0181435 A1
(43) Pub. Date: Jul. 16, 2009

(54) Method for Determining L-Serine, Gene Sequene, Vectors and Micro-Organisms

(76) Inventors: Lothar Eggeling, Juelich (DE); Petra Peters-Wendisch, Juelich (DE); Michael Stolz, Dueren (DE); Hermann Sahm, Juelich (DE)

Correspondence Address: JORDAN AND HAMBURG LLP, 122 EAST 42ND STREET, SUITE 4000, NEW YORK, NY, 10168, US

(21) Appl. No.: 12/083,662

(22) PCT Filed: Oct. 9, 2006
(86) PCT No.: PCT/DE2006/001756

§ 371 Date: Jul. 23, 2008

(30) Foreign Application Priority Data
Oct. 17, 2005 (DE) 10 2005 049 527.3

(51) Int. Cl. C12P 13/06; C12N 1/21; C12N 1/19; C12N 15/63; C07H 21/04

(52) U.S. Cl. 435/116; 435/252.3; 435/252.32; 435/254.2; 435/320.1; 536/23.7

(57) ABSTRACT

The folic acid concentration in amino acid producing organisms is reduced or completely removed in order to increase the production of L-serine. The enzymes and genes, which are involved in the biosynthesis of folic acid, can be completely excluded or at least reduced in activity. In a preferred embodiment, SEQ ID NOs: 1-7 are utilized.

The invention relates to a method for producing L-serine, and to gene sequences, vectors, and microorganisms suitable therefor.

### BACKGROUND OF THE INVENTION

The amino acid L-serine is used in human medicine, the pharmaceutical industry, the food industry, and animal nutrition.

It is known that amino acids are produced by fermenting strains of coryneform bacteria, in particular Corynebacterium glutamicum. Due to its great importance there are continuous efforts to improve the production process. Such improvements can relate to fermentation measures such as e.g. stirring and supplying with oxygen or the composition of nutrient media such as e.g. the sugar concentration during fermentation or processing for product form using e.g. ion exchange chromatography or the intrinsic properties of the microorganism itself.

Methods for mutagenesis, selection, and mutant selection are used for improving the properties of these microorganisms. In this manner strains are obtained that are resistant to antimetabolites or that are auxotrophic for metabolites that are meaningful in terms of regulator issues, and that produce L-amino acids and L-serine. Thus a coryneform bacterium is described that is resistant to azaserine or beta-(2-thienyl)-DL-alanine and produces L-serine (EP0943687). It is furthermore prior art that biosynthesis activities can be can be enhanced by means of genetic engineering methods and this leads to enhanced amino acid formation. Thus for example EP 0931833A2 describes that an increase in the biosynthesis enzyme phosphoserine-phosphatase and phosphoserine-transaminase is advantageous for L-serine formation. It is furthermore described that a gene that codes for D-3-phosphoglycerate-dehydrogenase can be used for L-serine formation (EP 0931833A2, PCT WO 93/12235).

Moreover it is known that, in addition to an increase in the activity of enzymes of the L-amino acid synthesis pathway, the decrease or even the exclusion of enzymes that are involved in degradation reactions for the L-amino acid can lead to an improvement in L-amino acid accumulation. Thus it has been found that excluding the enzyme L-serine dehydratase leads to higher L-serine accumulations (PCT/DE 2004/000248). Furthermore, a reduction in the activity of the enzyme serine hydroxymethyltransferase leads to reduced L-serine degradation (U.S. Pat. No. 6,596,516; Simic et al. (2002) Appl. Environ Microbiol. 68:3321-3327).

### BRIEF SUMMARY OF THE INVENTION

It is the object of the invention to provide a method, a microorganism suitable therefor, and vectors with which the production of L-serine can be increased. Moreover, a method, microorganisms, and vectors should be provided with which it is possible to attain an increase in the production of cysteine, tryptophan, and methionine.

Surprisingly, it was found that coryneform bacteria produce L-serine in an improved manner after modification or exclusion of the coding genes for folic acid synthesis. With the inventive method and the inventive bacteria and the inventive gene sequences for enzymes or regulators that catalyze folic acid synthesis or are involved in regulating folic acid synthesis it is also possible to produce L-serine with a yield that is substantially higher than that of strains not inventively modified. Table 2 portrays the extent of the increase in L-serine production.

The invention is described in general in the following:

In accordance with the invention, L-serine production and the production of cysteine, tryptophan, and methionine are increased in that the folic acid concentration is reduced in an organism that produces amino acid. The organism preferably already produces L-serine prior to the inventive modification.

For instance, the reduction in the folic acid concentration can be attained by reducing the synthesis of folic acid or by its degradation.

The reduction or prevention of synthesis of folic acid can be attained by directed or nondirected mutation of genes that are involved in the biosynthesis of folic acid.

Examples of mutations that lead to a reduction or exclusion of folic acid production are deletion mutation, insertion mutation, substitution mutation, or point mutation of genes that are involved in the biosynthesis of folic acid.

Furthermore, there can be a reduction or exclusion of folic acid production in genetically modified or unmodified genes of proteins that are involved in the biosynthesis of folic acid in that the expression of genes involved in the biosynthesis of folic acid is reduced or prevented.

It is possible to attain a reduction or exclusion of the expression of genes that are involved in the biosynthesis pathway of folic acid using modification of promoters, preferably weakening of promoters, particularly preferred exclusion of promoters such as signal structures, repressor genes, activators, operators, attenuators, ribosome binding sites or start codons, terminators, or furthermore by modifying, preferably weakening, particularly preferred exclusion or attenuating of regulators or the stability of the transcripts. Furthermore, regulatable promoters can be used, in particular weakened promoters can be used.

At the enzyme level, the activity of the enzymes involved in the biosynthesis of folic acid can be attained by reduction or exclusion of the catalytic activity and stability of the enzymes. The same effect can be attained by modifying the allosteric center or a feedback inhibition of the enzymes. One typical option for reducing or excluding the activity of the enzymes is protein modification, for instance by phosphorylation or adenylation. It is also possible to increase the proteolytic degradation of enzymes involved in the biosynthesis pathway of folic acid.

Typical enzymes, the activity of which can be reduced or excluded in the manner described, are GTP cyclohydrolase, neopterine triphosphate pyrophosphatase, neopterin aldolase, 6-hydroxymethylpterinpyrophosphokinase, 4-amino-4-deoxy-chorismate synthase, 4-amino-4-deoxy-chorismate lyase, pteroate synthase, folate synthase, and dihydrofolate reductase.

The subject-matter of the invention is also a vector that contains a tool that is suitable for causing inventive genetic modifications in a production organism.

The inventive vectors contain tools that are suitable for deleting the gene for synthesizing 4-amino-4-deoxy-chorismate synthase and/or 4-amino-4-deoxy-chorismate lyase.

The sequence for the deletion of the 4-amino-4-deoxy-chorismate synthase gene is depicted in SEQ ID NO: 1. SEQ ID NO: 2 illustrates the structure of a vector that bears SEQ ID NO: 1.

The sequence for the deletion of the 4-amino-4-deoxy-chorismate lyase gene is depicted in SEQ ID NO: 3. SEQ ID NO: 4 illustrates the structure of a vector that bears SEQ ID NO: 3.

The sequence for the deletion of the 4-amino-4-deoxy-chorismate synthase gene and the 4-amino-4-deoxy-chorismate lyase gene is depicted in SEQ ID NO: 5.

SEQ ID NO: 6 illustrates the structure of a vector that bears the SEQ ID NO: 5 for the deletion of the 4-amino-4-deoxy-chorismate synthase gene and the 4-amino-4-deoxy-chorismate lyase gene.

SEQ ID NO: 7 depicts a plasmid that is suitable for further increasing L-serine production. In FIG. 4 it is the plasmid designated pEC-T18mob2-serA^{fbr}CB-.

The structures responsible for the deletion can also be added to other vector models that are suitable for the specific organism. In addition to the frequently used cyclical factors, linear vectors or phages are also suitable for vectors.

### BRIEF DESCRIPTION OF THE DRAWINGS

The figures depict vectors that can be used for instance for the inventive modifications of the organisms producing L-serine.

FIG. 1: pK19mobsacB_pabAB corresponds to SEQ ID NO: 2;

FIG. 2: pK19mobsacB_pabC corresponds to SEQ ID NO: 4;

FIG. 3: pK19mobsacB_pabABC corresponds to SEQ ID NO: 6;

FIG. 4: pEC-T18mob2-serA^{fbr}CB corresponds to SEQ ID NO: 7.

The tools in accordance with SEQ ID NOs: 1, 3, and 5 are preferably added to vectors in L-serine production organisms that already produce L-serine prior to the modification.

All of the sequences indicated should also include variants that have 90%, preferably 95% homology.

Suitable organisms are for instance coryneform bacteria such as Corynebacterium glutamicum or Brevibacterium. It is also possible to use Enterobacteria, Bacillaceae, or yeasts types that exhibit a reduced folic acid concentration as production organisms.

### DETAILED DESCRIPTION OF THE INVENTION

The invention shall be described more precisely in the following.

The subject-matter of the invention is a method for fermentative production of L-serine using coryneform bacteria in which the genes that code for folic acid synthesis are modified, excluded, or changed in their expression or a deficiency of folic acid is caused in bacteria that naturally require folic acid or regulation of folic acid synthesis is influenced such that this deficiency in the folic acid occurs. Since folic acid synthesis proceeds from an intermediate of nucleotide synthesis and from an intermediate of the synthesis of aromatic amino acids, in principle these reactions can also be modified or excluded provided that nucleotides and aromatic amino acids themselves are available in sufficient quantities, as is the case for example with external supplementation. Moreover, the deficiency in folic acid can also be caused by the deletion or modification of regulators that control the expression of genes of folic acid or the metabolism pathways connected thereto.

These strains used preferably produce L-serine prior to the modification of the folic acid synthesis.

The term “modification” includes weakening folic acid synthesis genes and complete deletion of folic acid synthesis genes. This includes nondirected mutageneses and directed recombinant DNA techniques. Using these methods it is possible for instance to delete genes for folic acid synthesis in the chromosome. Suitable methods for this are described in Schäfer et al. (Gene (1994) 145: 69-73) and also Link et al. (J. Bacteriology (1998) 179: 6228-6237). It is also possible to delete only portions of the gene or even to exchange mutated fragments of genes. Thus the loss of or a reduction in folic acid synthesis activity is attained by depletion or exchange. One advantageous embodiment of the inventive method is for instance the inventively modified C. glutamicum strain ATCC13032DpykdsdaADpabABpserABC, which among other things bears a deletion in the pabAB gene.

Mutagenesis methods represent another option for weakening or excluding folic acid synthesis activity. Among these are nondirected methods that use chemical reagents such as e.g. N-methyl-N-nitro-N-nitrosoguanidine or even UV irradiation for mutagenesis, and a subsequent hunt in the desired microorganisms for a reduction in or loss of folic acid synthesis activity. Methods for initiating mutations and for mutant hunts are known in general and can be researched, inter alia, in Miller (A Short Course in Bacterial Genetics, A Laboratory Manual and Handbook for Escherichia coli and Related Bacteria (Cold Spring Harbor Laboratory Press, 1992)) or in the “Manual of Methods for General Bacteriology” from the American Society for Bacteriology (Washington D.C., USA, 1981).

In addition, the expression of genes from folic acid synthesis can be reduced by modifying the signal structures for gene expression. Signal structures are for instance repressor genes, activator genes, operators, promoters, attenuators, ribosome binding sites, the start codon, and terminators. One skilled in the art can find information regarding these e.g. in patent application WO 96/15246, in Boyd and Murphy (J. Bacteriol. 1988. 170: 5949), in Voskuil and Chambliss (Nucleic Acids Res. 1998. 26; 3548), in Jensen and Hammer (Biotechnol. Bioeng. 1998 58: 191), in Patek et al. (Microbiology (1996) 142: 1297) and in known textbooks in the field of genetics and molecular biology such as e.g. the textbook by Knippers (“Molekulare Genetik” [Molecular Genetics], 8th edition, Georg Thieme Verlag, Stuttgart, Deutschland, 2001) or that of Winnacker (“Gene und Klone” [Genes and Clones], VCH Verlagsgesellschaft, Weinheim, Deutschland, 1990). The promoter and regulation region that is disposed upstream of the structure gene can be mutated.

Using regulatable promoters it is also possible to reduce the expression during the course of the fermentative L-serine formation. In addition, however, it is possible to regulate the translation in that for instance the stability of the m-RNA is reduced. This can be attained in that the stability is weakened by additional and/or modified sequences at the 5′-end or 3′-end of the gene. Examples of this are described for genes from Bacillus subtilis (Microbiology (2001) 147; 1331-41) or yeast (Trends Biotechnol. 1994, 12:444-9). As described, it is furthermore possible to influence enzyme activity using intrinsic proteolytic activity (Mol. Microbiol. (2005) 57:576-91).

Moreover, genes can be used that code with less activity for the corresponding enzyme of the folic acid synthesis. Mutations that lead to a modification or reduction in the catalytic activity of enzyme proteins are known. Examples of this can be found in the works of Qiu and Goodman (J Biological Chemistry (1997) 272: 8611-8617), Sugimoto et al. (Bioscience Biotechnology and Biochemistry (1997) 61: 1760-1762), and Möckel (“Die Threonindehydratase aus Corynebacterium glutamicum: Auflebung der allosterischen Regulation und Struktur des Enzyms” [The Threonine Dehydratase from Corynebacterium glutamicum: Removing the Allosteric Regulation and Structure of the Enzyme], Reports from the Jülich Research Centre, Jül-2906, ISSN09442952, Jülich, Germany, 1994). Summarizing descriptions can be found in textbooks for the fields of genetics and molecular biology, such as e.g. by Hagemann (“Allgemeine Genetik” [General Genetics], Gustav Fischer Verlag, Stuttgart, 1986). Alternatively, moreover, it is possible to attain reduced expression and activity of the folic acid synthesis enzyme by modifying media composition and culturing. One skilled in the art can find instructions, inter alia, in Martin et al. (Bio/Technology 5, 137-146 (1987)), Guerrero et al. (Gene 138, 35-41 (1994)), Tsuchiya and Morinaga (Bio/Technology 6, 428-430 (1988)), Eikmanns et al. (Gene 102, 93-98 (1991)), European patent EP 0 472 869, U.S. Pat. No. 4,601,893, Schwarzer and Pühler (Bio/Technology 9, 84-87 (1991)), Reinscheid et al. (Applied and Environmental Microbiology 60, 126-132 (1994)), LaBarre et al. (Journal of Bacteriology 175, 1001-1007 (1993)), and in patent application WO 96/15246.

In this manner genes for folic acid synthesis of C. glutamicum can be expressed in a reduced manner or deleted or the enzyme activities can be reduced.

Furthermore, in addition to the induced deficiency of folic acid, it can be advantageous for the production of L-serine to strengthen, in particular to overexpress, individually or in combinations, one or a plurality of the genes selected from the group:
the serA gene that codes for the 3-phosphoglycerate-dehydrogenase;
the serC gene that codes for the phosphoserine-transaminase;
the serB gene that codes for the phosphoserine phosphatase;
   or to strengthen or overexpress, individually or in combinations, alleles of these genes, in particular
the serA genes that code for feedback-resistant 3-phosphoglycerate-dehydrogenase.

Moreover, in addition to the induced deficiency of folic acid, it can be advantageous for the production of L-serine to reduce or delete, individually or in combination, one or a plurality of the genes selected from the group:
the aecD gene that codes for the cystathionine lyase;
the metC gene that codes for the cystathionine lyase;
the sdaA gene that codes for the serine dehydratase;
the glyA gene that codes for the serine hydroxymethyltransferase;
the trpB gene that codes for the beta subunit of the tryptophan synthase;
the ilvA gene that codes for the threonine dehydratase;
the pyk gene that codes for the pyruvate kinase.

In the framework of the present invention, the inventive microorganisms include bacteria from the Corynebacterium or Brevibacterium genera that are modified using classical and/or genetic-molecular methods such that their metabolism flow is strengthened toward biosynthesis of amino acids or derivatives thereof. The present invention includes all of the already known amino acid production strains. Furthermore included in accordance with the invention are those production strains that one skilled in the art can produce according to current methods in analogy with experiences from other microorganisms, for instance Enterobacteria, Bacillaceae, and types of yeast. Furthermore included in accordance with the invention are also those amino acid production strains in which the degradation of L-serine is modified or weakened. This can occur for instance using directed genetic engineering modifications of enzymes that degrade L-serine or corresponding genes. A few microorganisms that are suitable in accordance with the invention are cited as examples in the following. This list shall not be restricting, however:

Corynebacterium glutamicum ATCC13032;
Corynebacterium acetoglutamicum ATCC 15806;
Corynebacterium acetoacidophilum ATCC13870;
Corynebacterium thermoaminogenes FERM BP-1539;
Brevibacterium flavum ATCC14067;
Brevibacterium lactofermentum ATCC13869; and
Brevibacterium divaricatum ATCC 14020.

The subject-matter of the present invention is also a pabAB and pabC gene SEQ ID NOs: 1, 3, or 5, that is characterized in that a part of the sequence was cut out by means of defined deletion so that only inactivated or weakened amino deoxychorismate synthase or amino deoxychorismate lyase activity can result. The pabAB and pabC gene sequences are preferably isolated from microorganisms from the Corynebacterium or Brevibacterium genus. A few of these more specifically identified microorganisms are listed here:

Corynebacterium glutamicum ATCC13032;
Corynebacterium acetoglutamicum ATCC15806;
Corynebacterium acetoacidophilum ATCC13870;
Corynebacterium thermoaminogenes FERM BP-1539;
Brevibacterium flavum ATCC14067;
Brevibacterium lactofermentum ATCC13869; and
Brevibacterium divaricatum ATCC14020.

### Example 1

### Structure of the Plasmid pK19mobsacBDpabAB

Using known methods, the pabAB gene from C. glutamicum is replaced in the chromosome by a pabAB gene shortened by 1734 bp (J. Bacteriol. (1997) 179:6228-37; Gene (1994) 145: 69-73). For constructing the vector used for the gene exchange, the primers listed in following were synthesized, and they were derived from the publicly accessible genome sequence (NCBI accession number YP_{—}225287; NC_{—}006985):

pabAB-del-A

(SEQ ID NO:8)

5′-CGGGATCCTCAGGCTCGCACGTTGGAGGG-3′
pabAB-del-B:

(SEQ ID NO:9)

5′-CCCATCCACTAAACTTAAACAAAACGTGAAAGAATCATAATT-3′
pabAB-del-C:

(SEQ ID NO:10)

5′-TGTTTAAGTTTAGTGGATGGGGAGTGGGAGGAAATCCGCGTT-3′
pabAB-del-D:

(SEQ ID NO:11)

5′-GTGGATCCGCCCAAAACACCACGGTGGCGT-3′

Primer pabAB-del-A begins 522 bp prior to the translation start and pabAB-del-D 436 bp behind the translation stop for the pabAB gene. The primer pabAB-del-B is 21 bp behind the translation start, the primer pabAB-del-C is 66 bp prior to the translation stop, and both have complementary linker regions, as indicated in Link et al. (J. Bacteriol. (1997) 179: 6228-37). PCR amplifications were performed in parallel with primer combination pabAB-del-A and pabAB-del-B and with primer combination pabAB-del-B and pabAB-del-C with chromosomal DNA from C. glutamicum ATCC13032. The PCR reaction was performed in 30 cycles in the presence of 200 μM deoxynucleotide triphosphates (dATP, dCTP, dGTP, dTTP), 600 nM each of the corresponding oligonucleotides, 100 ng chromosomal DNA of Corynebacterium glutamicum ATCC13032, 1/10 volume 10-fold reaction buffer, and 2.6 units of a heat-stable Taq/Pwo DNA polymerase mixture (Expand High Fidelity PCR System from Roche Diagnostics, Mannheim, Germany) in a thermocycler (PTC-100, MJ Research, Inc., Watertown, USA) under the following conditions:

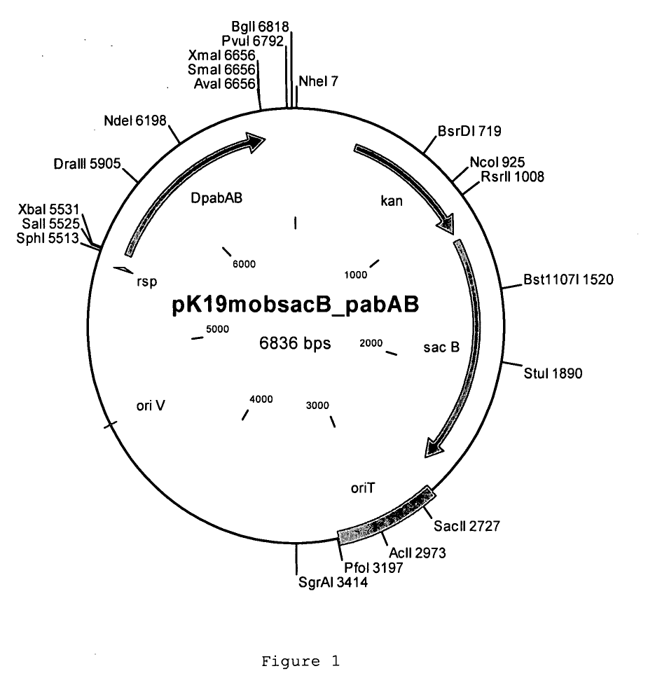

94° C. for 30 seconds, 50° C. for 30 seconds, and 72° C. for 40 seconds. The elongation step at 72° C. was extended after 10 cycles by five seconds per cycle. After the PCR reaction, the 563 bp fragment obtained from the 5′-flanking area and the 528 bp fragment from the 3′-flanking area were isolated from a 0.8% agarose gel using the QIAExII gel extraction kit (Qiagen) according to manufacturer instructions, and both fragments were used as templates in the second PCR with the primers pabAB-del-A and pabAB-del-D. Amplification occurred in 35 cycles in the presence of 200 μM deoxynucleotide triphosphates, 600 nM each of the corresponding oligonucleotides, 20 ng of the isolated template DNA from the first PCR, 1/10 volume 10-fold reaction buffer, and 2.6 units of the Taq/Pwo DNA polymerase mixture under the following conditions:
94° C. for 30 seconds, 50° C. for 30 seconds, and 72° C. for 80 seconds. The elongation step was extended after 10 cycles by five seconds each. After the PCR reaction, the 1122 bp DNA fragment obtained, which now contains the inactivated pabAB gene with a 1734 bp central deletion, was isolated from a 0.8% agarose gel and cloned in the vector pK19mobsacB (Gene 145: 69-73 (1994)). The resulting plasmid pK19mobsacBDpabAB (FIG. 1) was confirmed in terms of correctness by sequencing.

### Example 2

### Structure of the Plasmid pK19mobsacBDpabC

Analogous to the procedure described in Example 1, the vector pK19mobsacBDpabC suitable for the gene exchange was constructed for deleting the pabC gene of C. glutamicum. The primers required for this for PCR amplification were again derived from the publicly accessible genome sequence (NCBI accession number YP_{—}225288.1; NC_{—}006958). They are provided in the following:

pabC-del-A:

(SEQ ID NO:12)

5′-GAGGATCCAATCATTGCTGAGCTGCGCAG-3′
pabC-del-B:

(SEQ ID NO:13)

5′-CCCATCCACTAAACTTAAACAATCAACAACTGTGGGTGTTGA-3′
pabC-del-C:

(SEQ ID NO:14)

5′-TGTTTAAGTTTAGTGGATGGGTCGGTGAAGCCCTGGAATGAA-3′
pabC-del-D:

(SEQ ID NO:15)

5′-AGGGATCCGTGATGAGTCCGATCTCGGAA-3′

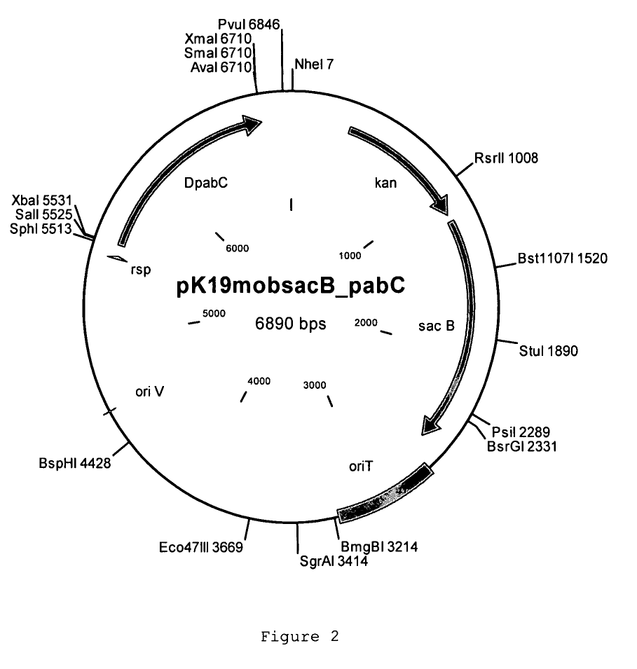

Primer pabC-del-A begins 500 bp prior to the translation start and pabC-del-D 500 bp behind the translation stop of the pabC gene. The primer pabC-del-B is 51 bp behind the translation start and pabC-del-C 48 bp prior to the translation stop. The last two primers each have complementary linker regions. In parallel, a 602 bp 5′ flanking area was amplified with the primary combination pabC-del-A and pabC-del-B and a 597 bp 3′ flanking area of the fragment to be deleted was amplified with the primary combination pabC-del-C und pabC-del-D. The PCR reaction was performed using the standard procedure, such as for instance in Example 1 with chromosomal DNA of C. glutamicum ATCC13032. After the PCR reaction, the DNA fragments obtained were isolated with the QIAExII gel extraction kit (Qiagen) and both fragments were used as templates in a further PCR. pabC-del-A and pabC-del-D were used as primers. The PCR reaction was performed using the standard procedure such as for instance in Example 1 with chromosomal DNA from C. glutamicum ATCC13032. After the PCR reaction, the 1178 bp DNA fragment obtained, which now contained the inactivated pabC gene with a 585 bp central deletion, was isolated from a 0.8% agarose gel and ligated with the vector pK19mobsacB (Schäfer et al. Gene 145: 69-73 (1994). The Escherichia coli strain DH5αmcr (Grant et al., Proceedings of the National Academy of Sciences of the United States of America USA (1990) 87: 4645-4649) was transformed with the ligation preparation. The plasmid pK19mobsacBDpabC obtained (FIG. 2) was examined for correctness using restriction digestion and sequencing.

### Example 3

### Structure of the Plasmids pK19mobsacBDpabABC

Analogous to the procedure described in Example 1, the vector pK19mobsacBDpabABC suitable for the gene exchange was constructed for deleting the pabABC gene of C. glutamicum. The primers required for this PCR amplification, derived from the publicly accessible genome sequence (NCBI accession number YP_{—}225287; YP_{—}225288.1; NC_{—}006958), are provided in the following:

pabABC-del-A:

(SEQ ID NO:8)

5′-CGGGATCCTCAGGCTCGCACGTTGGAGGG-3′
pabABC-del-13:

(SEQ ID NO:9)

5′-CCCATCCACTAAACTTAAACAAAACGTGAAAGAATCATAATT-3′
pabABC-del-C:

(SEQ ID NO:14)

5′-TGTTTAAGTTTAGTGGATGGGTCGGTGAAGCCCTGGAATGAA-3′
pabABC-del-D:

(SEQ ID NO:15)

5′-AGGGATCCGTGATGAGTCCGATCTCGGAA-3′

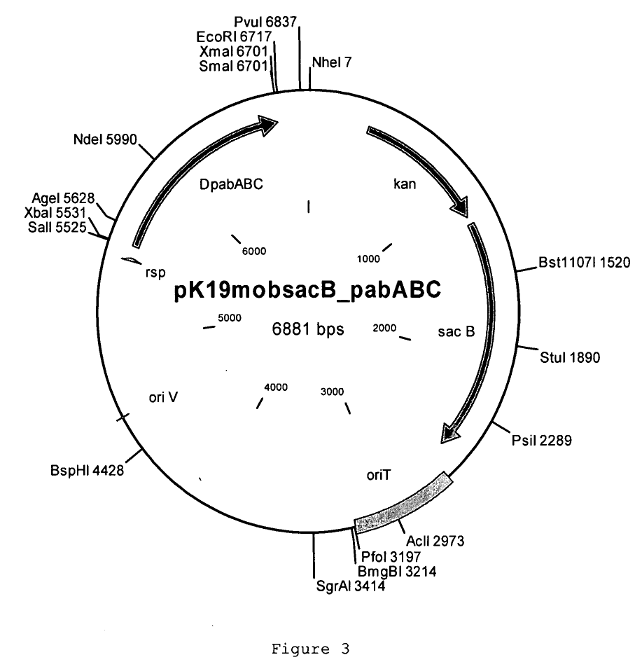

Primer pabABC-del-A begins 500 bp prior to the translation start and pabABC-del-D begins 500 bp behind the translation stop of the pabABC gene. Primer pabABC-del-B is 51 bp behind the translation start of pabAB and pabABC-del-C is 48 bp prior to the translation stop of pabC. The latter two primers each have complementary linker regions. In parallel, a 593 bp 5′ flanking area was amplified with the primary combination pabABC-del-A and pabABC-del-B and a 597 bp 3′ flanking area of the fragment to be deleted was amplified with the primary combination pabABC-del-C and pabABC-del-D. The PCR reaction was performed using the standard procedure, such as for instance in Example 1 with chromosomal DNA of C. glutamicum ATCC13032. After the PCR reaction, the DNA fragments obtained were isolated with the QIAExII gel extraction kit (Qiagen) and both fragments were used as templates in a further PCR. Primers pabABC-del-A and pabABC-del-D were used as primers. The PCR reaction was performed using the standard procedure such as for instance in Example 1 with chromosomal DNA from C. glutamicum ATCC13032. After the PCR reaction, the 1169 bp DNA fragment obtained, which contained the inactivated pabABC gene with a 2475 bp central deletion, was isolated from a 0.8% agarose gel and ligated with the vector pK19mobsacB (Schäfer et al. Gene 145: 69-73 (1994)). The Escherichia coli strain DH5αmcr (Grant et al., Proceedings of the National Academy of Sciences of the United States of America USA (1990) 87: 4645-4649) was transformed with the ligation preparation. The plasmid pK19mobsacBDpabC obtained (FIG. 3) was examined for correctness using restriction digestion and sequencing.

### Example 4

### Structure of the Folic Acid Auxotrophic Strains C. glutamicum DsdaADpabAB, C. glutamicum DsdaADpabC, C. glutamicum DsdaADpabABC

The plasmid pK19mobsacBDpabAB, or pK19mobsacBDpabC and pK19mobsacBDpabABC, was added to C. glutamicum DsdaA, which is described in patent application PCT/DE2004/000248, by means of electroporation, and selected for kanamycin resistance. Only those clones in which the plasmid was integrated into the chromosome using homologous recombination were kanamycin-resistant. In each case, one clone was selected that after examination demonstrated the saccharose sensitivity imparted by the plasmid (Gene (1994) 145: 69-73). This clone was cultivated in 50 ml BHI medium (brain-heart infusion medium, Difco Laboratories, Detroit, USA) without kanamycin or saccharose. Then 100 μl from 10-2, 10-3, and 10-4 dilutions of the culture were each plated on BHIS plates (BHI medium with 0.5 M sorbitol) with 10% (w/v) saccharose (FEMS Microbiol Lett. (1989) 53:299-303). The saccharose-resistant clones obtained were then tested for kanamycin sensitivity and verified using PCR analysis. The following primer combinations were used:

| | | |
|---|---|---|
| Deletion of pabAB: | | |
| pabAB-del-pr-u2 | 5′-TGGCTCACTTCGCTGGTCTTGTTG-3′ | (SEQ ID NO:16) |
| pabAB-del-pr-12 | 5′-GAATGGTTGCGGCGAGTGTCA-3′ | (SEQ ID NO:17) |
| Deletion of pabC: | | |
| pabC-del-pr-u2 | 5′-GTTGGGGGAGCAGGACGAGTGGT-3′ | (SEQ ID NO:18) |
| pabC-del-pr-12 | 5′-TACGCGCATCTGGAAGCCTGGTTA-3′ | (SEQ ID NO:19) |
| Deletion of pabABC: | | |
| pabABC-del-pr-u2 | 5′-CGTTCCGGCATCATTCTGGCTAAG-3′ | (SEQ ID NO:20) |
| pabABC-del-pr-12 | 5′-GCGACTCCGGGTTGTTCCTGATAA-3′ | (SEQ ID NO:21) |

The successful deletion resulted in a band of 1426 bp for the pabAB gene site, a band of 1663 bp for the pabC gene site, and a band of 1447 bp for the pabABC gene site. In this manner it was possible to obtain starting strain clones that have specific deletions in genes for folic acid synthesis.

These strains were designated C. glutamicum DsdaADpabAB, C. glutamicum DsdaADpabC, and C. glutamicum DsdaADpabABC.

These three strains were plated on the minimal medium CGXII (J Bacteriol. (1993) 175:5595-603) and on identical medium that contained the 1 mM folic acid. Table 1 provides the results.

TABLE 1
Folic acid requirement for the Corynebacterium glutamicum
mutants produced.

| | Growth | | | |
|---|---|---|---|---|
| | C. glu. | C. glu. | C. glu. | C. glu. |
| Folic acid | DsdaA | DsdaADpabAB | DsdaADpabC | DsdaADpabABC |
| 0 mM | + | − | − | − |
| 1 mM | + | + | + | + |

### Example 5

### Structure and L-Serine Formation of C. glutamicum DsdaADpabAB pserABC Strain

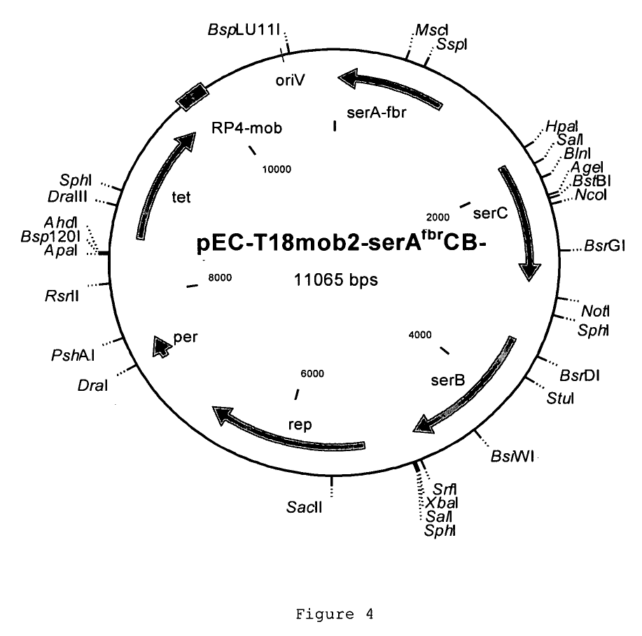

The plasmid pEC-T18mob2-serAfbrserCserB was added to the C. glutamicum DsdaADpabAB strain using electroporation. The plasmid (FIG. 4) comprises the vector pEC-T18mob2 (Curr. Microbiol. (2002) 45, 362-367), the corynebacterial genes serAfbr (Appl Microbiol Biotechnol. (2002) 60:437-41) and serC and serB (German patent application 10044 831.3). Tetracycline-resistant clones were tested using known standard methods for the presence and integrity of the plasmid pEC-T18mob2-serAfbrserCserB (Sambrook et al. (1989) Molecular Cloning, A Laboratory Manual, Cold Spring Harbor Laboratory Press) and a clone was designated C. glutamicum DsdaADpabAB pserABC.

For forming L-serine, the strain C. glutamicum DsdaADpabAB pserABC in complex medium (CgIII with 2% glucose, 5 μg/l tetracycline) was used and the fermentation medium CGXII (J Bacteriol (1993) 175: 5595-5603) was inoculated from it. The fermentation medium CGXII contains 0.1 or 1 mM folic acid. The strain C. glutamicum DsdaA pserABC was cultivated as a control in the same manner. At least two independent fermentations were performed for each. After cultivation for 30 hours at 30° C. on the rotation shaker at 120 rpm, the quantity of L-serine accumulated in the medium was measured. The amino acid concentration was analyzed by means of high pressure liquid chromatography (J Chromat (1983) 266: 471-482). The results of the fermentation are presented in Table 2. It is apparent that the use of the constructed and described mutants in folic acid synthesis represents a method that significantly improves L-serine formation.

TABLE 2
Accumulation of L-serine in the culture supernatant
of C. glutamicum DsdaADpabAB pserABC and C. glutamicum
DsdaA pserABC.

| Folic acid | | L-serin3 |
|---|---|---|
| (mM) | Strain | (mM) |
| 0.01 | C. glutamicum | 49 |
| | DsdaADpabAB pserABC | |
| 0.1 | C. glutamicum | 89 |
| | DsdaADpabAB pserABC | |
| 1.0 | C. glutamicum | 2 |
| | DsdaADpabAB pserABC | |
| 0.01 | C. glutamicum | 2 |
| | DsdaA pserABC | |
| 0.1 | C. glutamicum | 1 |
| | DsdaA pserABC | |
| 1.0 | C. glutamicum | 1 |
| | DsdaA pserABC | |

### Example 6

### Structure and L-Serine Formation of the C. glutamicum DpykDsdaADpabAB pserABC Strain

The plasmid pK19mobsacBDpyk (Arch Microbiol. (2004) 182:354-63) was added to the strain C. glutamicum 13032DsdaADpabABC by means of electroporation and selected for kanamycin-resistance. Only those clones in which the plasmid was integrated into the chromosomal pyk gene locus using homologous recombination were kanamycin-resistant. One clone was selected after examination demonstrated the saccharose sensitivity imparted by the plasmid and it was cultivated in 50 ml BHI medium (brain heart infusion medium, Difco Laboratories, Detroit, USA) without kanamycin and saccharose. Then 100 μl from 10-2, 10-3 and 10-4dilutions of the culture were each plated on BHIS plates (BHI medium with 0.5 M sorbitol) with 10% (w/v) saccharose. The saccharose-resistant clones obtained were then tested for kanamycin sensitivity and the successful deletion was verified using PCR analysis. The C. glutamicum DpykDsdaADpabAB strain could be obtained from the starting strain in this manner. As described in Example 5, this strain was transformed with pEC-T18mob2-serAfbrserCserB and the C. glutamicum DpykDsdaADpabAB pserABC strain was obtained. As described in Example 5, this strain was used for L-serine formation, in comparison to C. glutamicum DsdaADpabAB pserABC. Table 3 provides the results.

TABLE 3
Accumulation of L-serine in culture supernatant
of C. glutamicum DpykDsdaADpabAB pserABC and C. glutamicum
DsdaADpabAB pserABC.

| Folic acid | | L-serine |
|---|---|---|
| (mM) | Strain | (mM) |
| 0.1 | C. glutamicum | 89 |
| | DsdaADpabAB pserABC | |
| 0.1 | C. glutamicum | 98 |
| | DpykDsdaADpabAB pserABC | |
| 1.0 | C. glutamicum | 2 |
| | DpykDsdaADpabAB pserABC | |

1. Method for producing L-serine, comprising reducing folic acid concentration in an organism that produces an amino acid.

2. Method in accordance with claim 1, wherein said reducing of folic acid concentration comprises degrading of folic acid or reducing a synthesis of folic acid.

3. Method in accordance with either of claims 1 or 2, wherein said reducing of folic acid concentration comprises directed or nondirected mutation of genes involved in a biosynthesis of folic acid.

4. Method in accordance with claim 3, wherein said directed or nondirected mutation of genes comprises deletion mutation, insertion mutation, point mutation, and/or substitution mutation of said genes to reduce or prevent folic acid production.

5. Method in accordance with claims 1 or 2, wherein said reducing of folic acid concentration comprises reducing or preventing an expression of genes involved in a biosynthesis of folic acid.

6. Method in accordance with claim 5, wherein said reducing of expression of genes comprises changing or weakening or excluding promoters, signal structures, repressor genes, activators, operators, attenuators, ribosome binding sites, start codons, terminators, or regulators, or comprises reducing a stability of transcripts, or comprises using regulatable promoters.

7. Method in accordance with claim 3, wherein said directed or nondirected mutation of genes comprises changing genes coding for GTP cyclohydrolase, neopterine triphosphate pyrophosphatase, neopterin aldolase, 6-hydroxymethylpterinpyrophosphokinase, 4-amino-4-deoxy-chorismate synthase, 4-amino-4-deoxy-chorismate lyase, pteroate synthase, folate synthase, or dihydrofolate reductase.

8. Method in accordance with claim 7, wherein said changing comprises deleting the genes coding for 4-amino-4-deoxy-chorismate synthase.

9. Method in accordance with claim 8, wherein said deleting comprises deleting the genes coding for 4-amino-4-deoxy-chorismate with a DNS comprising SEQ ID NO: 1.

10. Method in accordance with claim 8, wherein said deleting comprises deleting the genes coding for 4-amino-4-deoxy-chorismate with a vector comprising SEQ ID NO: 2.

11. Method in accordance with claim 3, wherein said directed or nondirected mutation of genes comprises deleting a gene coding for 4-amino-4-deoxy-chorismate lyase.

12. Method in accordance with claim 11, wherein said deleting comprises deleting the gene coding for 4-amino-4-deoxy-chorismate lyase with a DNS comprising SEQ ID NO: 3.

13. Method in accordance with claim 11, wherein said deleting comprises deleting the gene coding for 4-amino-4-deoxy-chorismate lyase with a vector comprising SEQ ID NO: 4.

14. Method in accordance with claim 3, wherein said directed or nondirected mutation of genes comprises deleting a gene coding for 4-amino-4-deoxy-chorismate synthase and 4-amino-4-deoxy-chorismate lyase.

15. Method in accordance with claim 14, wherein said deleting comprises deleting the gene coding for 4-amino-4-deoxy-chorismate synthase and 4-amino-4-deoxy-chorismate lyase with a DNA sequence comprising SEQ ID NO: 5.

16. Method in accordance with claim 14, wherein said deleting comprises deleting the gene coding for 4-amino-4-deoxy-chorismate synthase and 4-amino-4-deoxy-chorismate lyase with a vector comprising SEQ ID NO: 6.

17. Method in accordance with claims 1 or 2, wherein said organism produces L-serine prior to the reducing of said folic acid concentration.

18. Method in accordance with claims 1 or 2, wherein the organism comprises a plasmid comprising SEQ ID NO: 7, said organism being a microorganism.

19. Method in accordance with claims 1 or 2, wherein said reducing of said folic acid concentration comprises weakening or preventing a catalytic activity of one or more enzymes involved in a biosynthesis of folic acid.

20. Method in accordance with claim 19, wherein said weakening or preventing comprises reducing a stability of said one or more enzymes.

21. Method in accordance with claim 19, wherein said weakening or preventing comprises changing an allosteric center of said one or more enzymes.

22. Method in accordance with claim 19, wherein said weakening or preventing comprises phosphorylation or adenylation of said one or more enzymes.

23. Method in accordance with claim 19, wherein said weakening or preventing comprises proteolytic degradation of said one or more enzymes.

24. Method in accordance with claim 19, wherein said one or more enzymes comprises at least one selected from the group consisting of: GTP cyclohydrolase, neopterine triphosphate pyrophosphatase, neopterin aldolase, 6-hydroxymethylpterinpyrophosphokinase, 4-amino-4-deoxy-chorismate synthase, 4-amino-4-deoxy-chorismate lyase, pteroate synthase, folate synthase, and dihydrofolate reductase.

25. Method in accordance with claim 1, wherein said reducing folic acid concentration comprises strengthening or overexpressing, individually or in combinations, at least one gene selected from the group consisting of
a serA gene that codes for a 3-phosphoglycerate-dehydrogenase,
a serC gene that codes for a phosphoserine-transaminase, and
a serB gene that codes for a phosphoserine phosphatase.

26. Method in accordance with claim 25, wherein said strengthening or overexpressing comprises strengthening or overexpressing alleles of said at least one gene.

27. Method in accordance with claim 1, wherein said reducing of folic acid concentration comprises reducing or eliminating an activity of at least one gene selected from the group consisting of:
an aecD gene that codes for a cystathionine lyase,
a metC gene that codes for a cystathionine lyase,
an sdaA gene that codes for a serine dehydratase,
a glyA gene that codes for a serine hydroxymethyltransferase,
a trpB gene that codes for a beta subunit of a tryptophan synthase,
an ilvA gene that codes for a threonine dehydratase, and
a pyk gene that codes for a pyruvate kinase.

28. Method in accordance with claim 1 or 2, wherein said organism is a methanol-utilizing bacterium or a yeast.

29. Method in accordance with claim 1 or 2, wherein said organism is a Corynebacterium glutamicum ATCC13032, Corynebacterium acetoglutamicum ATCC15806, Corynebacterium acetoacidophilum ATCC13870, Corynebacterium thermoaminogenes FERM BP-1539, Brevibacterium flavum ATCC14067, Brevibacterium lactofermentum ATCC 13869, or Brevibacterium divaricatum ATCC14020.

30. Method according to claim 1 or 2, further comprising synthesizing cysteine, tryptophan, or methionine.

31. Gene sequence comprising at least 85% homology to SEQ ID NOs: 1, 3, or 5.

32. Gene sequence in accordance with claim 31, wherein said gene sequence is identical to SEQ ID NOs: 1, 3, or 5.

33. Vector comprising a gene sequence having at least 85% homology to SEQ ID NOs: 2, 4, or 6.

34. Vector in accordance with claim 33, wherein said gene sequence is identical to SEQ ID NOs: 2, 4, or 6.

35. Vector comprising a sequence having at least 85% homology to SEQ ID NO: 7.

36. Vector in accordance with claim 35, wherein said sequence is identical to SEQ ID NO: 7.

37. Organism which is modified in accordance with the method according to claim 1 or 2, said organism being a microorganism.

38. Organism according to claim 37, wherein said organism is a Corynebacterium, Brevibacterium, Bacillacceae, Enterobacterium, or yeast.

39. Organism in accordance with claim 38, wherein said organism is a Corynebacterium glutamicum ATCC 13032, Corynebacterium cetoglutamicum ATCC15806, Corynebacterium acetoacidophilum ATCC13870, Corynebacterium thermoaminogenes FERM BP-1539, Brevibacterium flavum ATCC 14067, Brevibacterium lactofermentum ATCC 13869, or Brevibacterium divaricatum ATCC 14020.

40. Method in accordance with claim 26, wherein said at least one gene comprises a serA gene that codes for feedback-resistant 3-phosphoglycerate-dehydrogenase.

41. Method in accordance with claim 1 or 2, wherein said organism is a Corynebacterium, Brevibacterium, Bacillaceae, or Enterobacterium.


SEQUENCE LISTING (21 sequences)

SEQ ID NO: 1 - DNA, 1122 bp, Corynebakterium glutamicum
cgggatcctcaggctcgcacgttggagggtgctcgcattcttgctgagcgtctgactgcttctgatgcgaaggccgctggcgtggatgtcttgaccggtggcactgatgtgcacttggttttggctgatctgcgtaactcccagatggatggccagcaggcggaagatctgctgcacgaggttggtatcactgtgaaccgtaacgcggttcctttcgatcctcgtccaccaatggttacttctggtctgcgtattggtactcctgcgctggctacccgtggtttcgatattcctgcattcactgaggttgcagacatcattggtactgctttggctaatggtaagtccgcagacattgagtctctgcgtggccgtgtagcaaagcttgctgcagattacccactgtatgagggcttggaagactggaccatcgtctaagtttttctttgagttttcatatgtagaaggcatcgtcggcttcggcctggcggtgcttttctcgttgttttgtggttttgtcagaggatgtcatgcgcgttttaattattgataattatgattctttcacgttttgtttaagtttagtggatggggagtgggaggaaatccgcgttaaatcacggcctctgctgaatttgtttggggttgaattcccatgacgtacctcgtgtgggacggtgcaacactcgtagaaggcgcgctggaatcaacacccacagttgttgattcctacctagccaaagaccaccgcgtggtgcgctgggatcttcatgaacagcgcttcgccactagcgtggacgtggacccgtgggattttctccacgcagtaagggaagcaattccacgccagggctcatggtttcccaaagttgaatggcatggcgatgatcttttcgcagtcaatattcgcccggcaccaacactgcgaaaggccacatcattgtggctttccgaagacccagatccacgcacacagccaaccattaaaggcccagacctagatgtgcttgctcaccttcgcagtcgcgccaacgataacggctgcgatgatgcgctgttgatcagcgcggatgggttcattctggaagctgccaacgccaccgtggtgttttgggcggatcc

SEQ ID NO: 2 - DNA, 6836 bp, Corynebakterium glutamicum
cgataagctagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgctagaggatcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagtttctgaatttgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccgagcttcctgctgaacatcaaaggcaagaaaacatctgttgtcaaagacagcatccttgaacaaggacaattaacagttaacaaataaaaacgcaaaagaaaatgccgatgggtaccgagcgaaatgaccgaccaagcgacgcccaacctgccatcacgagatttcgattccaccgccgccttctatgaaaggttgggcttcggaatcgttttccgggacgccctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgccccgctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagggctggatgatcctccagcgcggggatctcatgctggagttcttcgcccaccccaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccgcccaaaacaccacggtggcgttggcagcttccagaatgaacccatccgcgctgatcaacagcgcatcatcgcagccgttatcgttggcgcgactgcgaaggtgagcaagcacatctaggtctgggcctttaatggttggctgtgtgcgtggatctgggtcttcggaaagccacaatgatgtggcctttcgcagtgttggtgccgggcgaatattgactgcgaaaagatcatcgccatgccattcaactttgggaaaccatgagccctggcgtggaattgcttcccttactgcgtggagaaaatcccacgggtccacgtccacgctagtggcgaagcgctgttcatgaagatcccagcgcaccacgcggtggtctttggctaggtaggaatcaacaactgtgggtgttgattccagcgcgccttctacgagtgttgcaccgtcccacacgaggtacgtcatgggaattcaaccccaaacaaattcagcagaggccgtgatttaacgcggatttcctcccactccccatccactaaacttaaacaaaacgtgaaagaatcataattatcaataattaaaacgcgcatgacatcctctgacaaaaccacaaaacaacgagaaaagcaccgccaggccgaagccgacgatgccttctacatatgaaaactcaaagaaaaacttagacgatggtccagtcttccaagccctcatacagtgggtaatctgcagcaagctttgctacacggccacgcagagactcaatgtctgcggacttaccattagccaaagcagtaccaatgatgtctgcaacctcagtgaatgcaggaatatcgaaaccacgggtagccagcgcaggagtaccaatacgcagaccagaagtaaccattggtggacgaggatcgaaaggaaccgcgttacggttcacagtgataccaacctcgtgcagcagatcttccgcctgctggccatccatctgggagttacgcagatcagccaaaaccaagtgcacatcagtgccaccggtcaagacatccacgccagcggccttcgcatcagaagcagtcagacgctcagcaagaatgcgagcaccctccaacgtgcgagcctgaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcg

SEQ ID NO: 3 - DNA, 1178 bp, Corynebakterium glutamicum
gaggatccaatcattgctgagctgcgcagtaatcctaaagatcgtgcagaaaacttgatgatcgtggatttggtccgcaacgacttagcccgcggcgctttgcccaccacagttaaaacatccaagcttttcgacgtcgaaacctacgccacagtccaccaacttgtcagcaccgtctctgcagagttggggccacgcagtccgattgagtgcgtgcgcgcagcattccccggtggttcgatgactggtgccccaaagctgcgcaccatggagatcatcgatgagctggaggcagctcctcgcggtatttactcaggtggcttgggatatttttccctcgacggcgcagttgatctctccatggtgatcagaactctcgtcatccagaacaatcacgtggagtacggagtgggcggtgcacttcttgctctgtctgatccggaggctgagtgggaggaaatccgcgttaaatcacggcctctgctgaatttgtttggggttgaattcccatgacgtacctcgtgtgggacggtgcaacactcgtagaaggcgcgctggaatcaacacccacagttgttgattgtttaagtttagtggatgggtcggtgaagccctggaatgaaaaattgcgcccaaccatttttctgtgaggaaaaggttgagcgcagtctttgtgaagatttagtcttcggtagtttcttcagtttctttctttgcggtttcgcgaccttcagcaagctgtgtgcgtgcggtgcgcaaccattctggctggttttccagaagtgccttgatctcggcggtggtcagtggtttgtccatgtcgttctttttcagagccgcgatggtgatgcccaatttttgggcgaccactggacgagggtgtgggccctcgcggcgtagggtttggagccactccggtgggttttcctgcaggttcttgaactcttggtgagtcaatgcacctgtttggaactcctctggcgtggcgggcaaaaacagtccgagcttcttagcggcggtctgtggcttcatagccctgcccgatggctggcgtacagattcttcgttcacgcccacaacggtagcattgttttcatgctgaccttaagtttcatcactggcacggagccaggaaagtggtttacccgattccgagatcggactcatcacggatccct

SEQ ID NO: 4 - DNA, 6890 bp, Corynebakterium glutamicum
cgataagctagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgctagaggatcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagtttctgaatttgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccgagcttcctgctgaacatcaaaggcaagaaaacatctgttgtcaaagacagcatccttgaacaaggacaattaacagttaacaaataaaaacgcaaaagaaaatgccgatgggtaccgagcgaaatgaccgaccaagcgacgcccaacctgccatcacgagatttcgattccaccgccgccttctatgaaaggttgggcttcggaatcgttttccgggacgccctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgccccgctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagggctggatgatcctccagcgcggggatctcatgctggagttcttcgcccaccccaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccaatcattgctgagctgcgcagtaatcctaaagatcgtgcagaaaacttgatgatcgtggatttggtccgcaacgacttagcccgcggcgctttgcccaccacagttaaaacatccaagcttttcgacgtcgaaacctacgccacagtccaccaacttgtcagcaccgtctctgcagagttggggccacgcagtccgattgagtgcgtgcgcgcagcattccccggtggttcgatgactggtgccccaaagctgcgcaccatggagatcatcgatgagctggaggcagctcctcgcggtatttactcaggtggcttgggatatttttccctcgacggcgcagttgatctctccatggtgatcagaactctcgtcatccagaacaatcacgtggagtacggagtgggcggtgcacttcttgctctgtctgatccggaggctgagtgggaggaaatccgcgttaaatcacggcctctgctgaatttgtttggggttgaattcccatgacgtacctcgtgtgggacggtgcaacactcgtagaaggcgcgctggaatcaacacccacagttgttgattgtttaagtttagtggatgggtcggtgaagccctggaatgaaaaattgcgcccaaccatttttctgtgaggaaaaggttgagcgcagtctttgtgaagatttagtcttcggtagtttcttcagtttctttctttgcggtttcgcgaccttcagcaagctgtgtgcgtgcggtgcgcaaccattctggctggttttccagaagtgccttgatctcggcggtggtcagtggtttgtccatgtcgttctttttcagagccgcgatggtgatgcccaatttttgggcgaccactggacgagggtgtgggccctcgcggcgtagggtttggagccactccggtgggttttcctgcaggttcttgaactcttggtgagtcaatgcacctgtttggaactcctctggcgtggcgggcaaaaacagtccgagcttcttagcggcggtctgtggcttcatagccctgcccgatggctggcgtacagattcttcgttcacgcccacaacggtagcattgttttcatgctgaccttaagtttcatcactggcacggagccaggaaagtggtttacccgattccgagatcggactcatcacggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcg

SEQ ID NO: 5 - DNA, 1169 bp, Corynebakterium glutamicum
cgggatcctcaggctcgcacgttggagggtgctcgcattcttgctgagcgtctgactgcttctgatgcgaaggccgctggcgtggatgtcttgaccggtggcactgatgtgcacttggttttggctgatctgcgtaactcccagatggatggccagcaggcggaagatctgctgcacgaggttggtatcactgtgaaccgtaacgcggttcctttcgatcctcgtccaccaatggttacttctggtctgcgtattggtactcctgcgctggctacccgtggtttcgatattcctgcattcactgaggttgcagacatcattggtactgctttggctaatggtaagtccgcagacattgagtctctgcgtggccgtgtagcaaagcttgctgcagattacccactgtatgagggcttggaagactggaccatcgtctaagtttttctttgagttttcatatgtagaaggcatcgtcggcttcggcctggcggtgcttttctcgttgttttgtggttttgtcagaggatgtcatgcgcgttttaattattgataattatgattctttcacgttttgtttaagtttagtggatgggtcggtgaagccctggaatgaaaaattgcgcccaaccatttttctgtgaggaaaaggttgagcgcagtctttgtgaagatttagtcttcggtagtttcttcagtttctttctttgcggtttcgcgaccttcagcaagctgtgtgcgtgcggtgcgcaaccattctggctggttttccagaagtgccttgatctcggcggtggtcagtggtttgtccatgtcgttctttttcagagccgcgatggtgatgcccaatttttgggcgaccactggacgagggtgtgggccctcgcggcgtagggtttggagccactccggtgggttttcctgcaggttcttgaactcttggtgagtcaatgcacctgtttggaactcctctggcgtggcgggcaaaaacagtccgagcttcttagcggcggtctgtggcttcatagccctgcccgatggctggcgtacagattcttcgttcacgcccacaacggtagcattgttttcatgctgaccttaagtttcatcactggcacggagccaggaaagtggtttacccgattccgagatcggactcatcacggatccct

SEQ ID NO: 6 - DNA, 6881 bp, Corynebakterium glutamicum
cgataagctagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgctagaggatcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagtttctgaatttgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccgagcttcctgctgaacatcaaaggcaagaaaacatctgttgtcaaagacagcatccttgaacaaggacaattaacagttaacaaataaaaacgcaaaagaaaatgccgatgggtaccgagcgaaatgaccgaccaagcgacgcccaacctgccatcacgagatttcgattccaccgccgccttctatgaaaggttgggcttcggaatcgttttccgggacgccctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgccccgctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagggctggatgatcctccagcgcggggatctcatgctggagttcttcgcccaccccaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatcctcaggctcgcacgttggagggtgctcgcattcttgctgagcgtctgactgcttctgatgcgaaggccgctggcgtggatgtcttgaccggtggcactgatgtgcacttggttttggctgatctgcgtaactcccagatggatggccagcaggcggaagatctgctgcacgaggttggtatcactgtgaaccgtaacgcggttcctttcgatcctcgtccaccaatggttacttctggtctgcgtattggtactcctgcgctggctacccgtggtttcgatattcctgcattcactgaggttgcagacatcattggtactgctttggctaatggtaagtccgcagacattgagtctctgcgtggccgtgtagcaaagcttgctgcagattacccactgtatgagggcttggaagactggaccatcgtctaagtttttctttgagttttcatatgtagaaggcatcgtcggcttcggcctggcggtgcttttctcgttgttttgtggttttgtcagaggatgtcatgcgcgttttaattattgataattatgattctttcacgttttgtttaagtttagtggatgggtcggtgaagccctggaatgaaaaattgcgcccaaccatttttctgtgaggaaaaggttgagcgcagtctttgtgaagatttagtcttcggtagtttcttcagtttctttctttgcggtttcgcgaccttcagcaagctgtgtgcgtgcggtgcgcaaccattctggctggttttccagaagtgccttgatctcggcggtggtcagtggtttgtccatgtcgttctttttcagagccgcgatggtgatgcccaatttttgggcgaccactggacgagggtgtgggccctcgcggcgtagggtttggagccactccggtgggttttcctgcaggttcttgaactcttggtgagtcaatgcacctgtttggaactcctctggcgtggcgggcaaaaacagtccgagcttcttagcggcggtctgtggcttcatagccctgcccgatggctggcgtacagattcttcgttcacgcccacaacggtagcattgttttcatgctgaccttaagtttcatcactggcacggagccaggaaagtggtttacccgattccgagatcggactcatcacggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcg

SEQ ID NO: 7 - DNA, 11065 bp, Corynebakterium glutamicum
gaattgatccccctagagccggagacgtgaataaaattcgcagctcattccatcagcgtaaacgcagctttttgcatggtgagacacctttgggggtaaatctcacagcatgaatctctgggttagatgactttctgggtgggggagggtttagaatgtttctagtcgcacgccaaaacccggcgtggacacgtctgcagccgacgcggtcgtgcctgttgtagacggacattcctagtttttccaggagtaacttgtgagccagaatggccgtccggtagtcctcatcgccgataagcttgcgcagtccactgttgacgcgcttggagatgcagtagaagtccgttgggttgacggacctaaccgcccagaactgcttgatgcagttaaggaagcggacgcactgctcgtgcgttctgctaccactgtcgatgctgaagtcatcgccgctgcccctaacttgaagatcgtcggtcgtgccggcgtgggcttggacaacgttgacatccctgctgccactgaagctggcgtcatggttgctaacgcaccgacctctaatattcactccgcttgtgagcacgcaatttctttgctgctgtctactgctcgccagatccctgctgctgatgcgacgctgcgtgagggcgagtggaagcggtcttctttcaacggtgtggaaattttcggaaaaactgtcggtatcgtcggttttggccacattggtcagttgtttgctcagcgtcttgctgcgtttgagaccaccattgttgcttacgatccttacgctaaccctgctcgtgcggctcagctgaacgttgagttggttgagttggatgagctgatgagccgttctgactttgtcaccattcaccttcctaagaccaaggaaactgctggcatgtttgatgcgcagctccttgctaagtccaagaagggccagatcatcatcaacgctgctcgtggtggccttgttgatgagcaggctttggctgatgcgattgagtccggtcacattcgtggcgctggtttcgatgtgtactccaccgagccttgcactgattctcctttgttcaagttgcctcaggttgttgtgactcctcacttgggtgcttctactgaagaggctcaggatcgtgcgggtactgacgttgctgattctgtgctcaaggcgctggctggcgagttcgtggcggatgctgtgaacgtttccggtggtcgcgtgggcgaagaggttgctgtgtggatggatctggctctaaggatcgggtaccgagctcgaattaattcgagctcggtacccgaccacccacagccaccgtaatcagtagccacggtcacgccaatagaactcagatcaattgtgccgatgtgctgctatttagttctgcaccctcaaggccagcattcaaccccgctagagggtgaaaatgctggcctttaaggcattaaaaatcccacaataagtggactggtgcacagtttagcaaagtttgtgatgcacgcgacaaggatgggtgccgagtagctttccccatgcaattcgccaccctataacgcaacctcaggggatattaacaacctagaaattgaaaactttgcaaaactttgagctacccccaaattggtggctggtcaactaatccccgcgttttcaatagttcggtgtcgccagttttgggcgtttttcatcgtttgggagactgcgtgaagaatctagggtgctaggaactgacagcttcagggttatagttgttgggtcagatcgttaacgatccctggcccttttacttccaagcgcagaaagttgcccgaagacatgaccgacttccccaccctgccctctgagttcatccctggcgacggccgtttcggctgcggaccttccaaggttcgaccagaacagattcaggctattgtcgacggatccgcatccgtcatcggtacctcacaccgtcagccggcagtaaaaaacgtcgtgggttcaatccgcgagggactctccgacctcttctcccttccagaaggctacgagatcatcctttccctaggtggtgcgaccgcattctgggatgcagcaaccttcggactcattgaaaagaagtccggtcacctttctttcggtgagttctcctccaagttcgcaaaggcttctaagcttgctccttggctcgacgagccagagatcgtcaccgcagaaaccggtgactctccggccccacaggcattcgaaggcgccgatgttattgcatgggcacacaacgaaacctccactggcgccatggttccagttcttcgccccgaaggctctgaaggctccctggttgccattgacgcaacctccggcgctggtggactgccagtagacatcaagaactccgatgtttactacttctccccacagaagtgcttcgcatccgacggtggcctgtggcttgcagcgatgagcccagcagctctcgagcgcatcgagaagatcaacgcttccgatcgcttcatccctgagttcctcaacctgcagaccgcagtggataactccctgaagaaccagacctacaacaccccagctgttgctaccttgctgatgctggacaaccaggtcaagtggatgaactccaacggcggcctggatggaatggttgctcgcaccacagcaagctcctccgccctgtacaactgggctgaggctcgcgaggaggcatccccatacgtggcagatgcagctaagcgctccctcgttgtcggcaccatcgacttcgatgactccatcgacgcagcagtgatcgctaagatactgcgcgcaaacggcatcctggacaccgagccttaccgcaagctgggacgcaaccagctgcgcatcggtatgttcccagcgatcgattccaccgatgtggaaaagctcaccggagcaatcgacttcatcctcgatggcggttttgcaaggaagtaatacccccactttgaaaaacaccccgtacagttacaccagtacggggtgttttttagttaagcttgggtgatcacttaacccagctagaaggagtcaacatgcgggaaagcggccgcgttgatgatccttggggttacggcaactccctggagtgggcaacctcctgccctcctcctcgccacaacttcgcatccttgcctcgtatccgctccgagcgccctgcgttcgagctgcactacccgcacatgattgaacgcatgcgcgcagaggcacacactggacatcacgatgatattaatgctccagaattgggtaccgccccagcccttgcatctgactccagccgctaaaagcgtctgatttaagtcggtacctgactaaataagcaccagccccagcagagataatctgccggggctggtgcttttcatattccgacttggggcacccctgaatacatctcacccaattccccataactagacaattgcccagcaacgactgataagtctccaatgtcgtgttccgcgctcagacatgagacaattgttgccgtgactgaactcatccagaatgaatcccaagaaatcgctgagctggaagccggccagcaggttgcattgcgtgaaggttatcttcctgcggtgatcacagtgagcggtaaagaccgcccaggtgtgactgccgcgttctttagggtcttgtccgctaatcaggttcaggtcttggacgttgagcagtcaatgttccgtggctttttgaacttggcggcgtttgtgggtatcgcacctgagcgtgtcgagaccgtcaccacaggcctgactgacaccctcaaggtgcatggacagtccgtggtggtggagctgcaggaaactgtgcagtcgtcccgtcctcgttcttcccatgttgttgtggtgttgggtgatccggttgatgcgttggatatttcccgcattggtcagaccctggcggattacgatgccaacattgacaccattcgtggtatttcggattaccctgtgaccggcctggagctgaaggtgactgtgccggatgtcagccctggtggtggtgaagcgatgcgtaaggcgcttgctgctcttacctctgagctgaatgtggatattgcgattgagcgttctggtttgctgcgtcgttctaagcgtctggtgtgcttcgattgtgattccacgttgatcactggtgaggtcattgagatgctggcggctcacgcgggcaaggaagctgaagttgcggcagttactgagcgtgcgatgcgcggtgagctcgatttcgaggagtctctgcgtgagcgtgtgaaggcgttggctggtttggatgcgtcggtgatcgatgaggtcgctgccgctattgagctgacccctggtgcgcgcaccacgatccgtacgctgaaccgcatgggttaccagaccgctgttgtttccggtggtttcatccaggtgttggaaggtttggctgaggagttggagttggattatgtccgcgccaacactttggaaatcgttgatggcaagctgaccggcaacgtcaccggaaagatcgttgaccgcgctgcgaaggctgagttcctccgtgagttcgctgcggattctggcctgaagatgtaccagactgtcgctgtcggtgatggcgctaatgacatcgatatgctctccgctgcgggtctgggtgttgctttcaacgcgaagcctgcgctgaaggagattgcggatacttccgtgaaccacccattcctcgacgaggttttgcacatcatgggcatttcccgcgacgagatcgatctggcggatcaggaagacggcactttccaccgcgttccattgaccaatgcctaaagattcgcttctcgacgcccacctcctcctcaaggcccgggctagcgacgggccacatagcgaggatccttcggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgataagctagatccccatcaatcctgcctatttgccacgtttaacaaggtagttaagcgttcatttacgaagaaaacacgataagctgcacaaatacctgaaaaagttgaacgccccgtgagcgggaactcacagggcgtcggctaacccccagtcatcagctgggagaaagcactcaagacatgactctagccgatccgcaggacacagtcacagctagcgcgtggaaattgtccgccgatctgttcgacacccaccccgaagctatgcgctgcggctcacgcggctggacggcagaagatcgccgcgaactgctcgctcacctgggacgcgaaagcttccagggcagcaagacaagagatttcgcgagcgcctggattaaaaacccggataccggcgaaacccaaccaaagctctaccgggctggctcaaaagcgctgacgcggtgccagtacgttgcgctgacgcacgcgcaacatgccgcggtgatcgtgcttgacatcgatgtgcccagccaccaggccggcgggaagattgagcacgtaaacccgcaggtctacgcgattttagagaaatgggcacgcctagaaaaagcgccggcttggatcggcgtgaatccgctgagcgggaaatgccagctcatctggctcattgacccggtgtatgccgcagcaggtaaaaccagcccaaatatgcgcctgctggctgcaacgacggaagaaatgactcgtgttttcggcgctgaccaggctttttcgcataggctgagccggtggccgctgcacgtctcagacgatccgacagcctataaatggcactgccagcatgatcgtgtggatcggctggccgacctaatggagattgctcgaacgatgaccggatcacagaagccgaaaaagtacattgagcaggacttttccagcggacgcgcccgcattgaagcggcacaacgcgccaccgcagaagccaaggcgctagcgattttggacgcgagcctgccgagcgccctggacgcgtccggcgacctgatcgacggcgtgcgagtgctctggacaaatccagagcgagcgcgcgacgagaccgcgtttcgccacgcgttgaccgtgggataccagctcaaagctgctggtgagcgcctaaaagatgccaagatcatcgacgcgtatgaagtggcgtacaacgttgcccaggcggtcggtgcagacggccgggagccggatcttcccgccatgcgtgatcgcctgacgatggcgcgtcgtgtgcgcggctacgtggctaaaggccagccagtcgtccctgctcgtcgggtggaaacgcagagcagccgagggcggaaagctctagcgacgatggggcgacggggcgcagctacatcgaatgcacgcagatgggctgacccagaaagtaagtatgcgcaggagacgcgacagcgattagcggaagcaaacaaacgccgagaaatgacaggcgagttgctcgaacttcgcgtcaaaactgcgatcctggatgcccgttctcaatcggttgctgatccctcgactcgtgagcttgcaggcgaactaggtgtcagtgaaaggcgcatccaacaagtcagaaaggcacttggaatggaagctaaacgcggccgtccacgggctgaaaactaataaacgaaacaccgtcagcagaaaacggttcccccctttaggggtcccgtccttgctctggctctcacttgccctcaccctccgctatccacgggctgaaaactaataaacgaaacaccgtcagcagaaaacggttccccccctttagggtgtctcgctcctagctctgatccctccccggttcctccccggcctgatttttaaggggggctcacgctgtcggcagagaacggttccccgccttctgctctggctcttcctcgactccctccccctcaaaaatctcctcgagatcctggagacctttttggagctagcgcgttgctgcttcgcaccaacttgctcatgatgattttcatttttgcttgtgtgcttttttgggttgaaccctccaaagaggggaaaccaggggcacacctcatgcactaaagtgccgcttcgctggtcagggtgaaatcacctggaaaaaaagtgcggtaaccgctgcgcttggcgttttttctgggcaagaagtctcgcaggttttcgcaggagtgccggaagaaattatcagaattggggctagaatttttaacgaacgttcgttataatggtgtcatgaccttcacgacgaagtaccaaaactggcctgaagcatcagcggtggatctctccgatgtcgcgctggagtccgacgcactcgatgccgccgtcgatttaaaaacggtgatcggatttttccgcgccctcgatacgacagacgcgccagcatcacgcgactgggcaagtgccgcgagcgacctagaaacgcttgtggccgaccttgaagagctggccgacgagctgcgtgctcggcagcgccaggaggacgcgcagtagtggaggatcgcatcagctgcgcctactgcggtggcctgatcccaccccggcctgacccacgaggacggcgcgcaaaatactgctcagacgcgtgtcgtgccgcagccagccgcgagcgcgccaacaagcgccacgcccaggaggtcgaagccgcacgtcatctagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaactccttcgtcggtgtcgtcgccggatggtctgcggtggtgctcagcgtggagacgcgcaccgtcacggaccgcgtgtagtgcgtggcggaaacttcttgcgtttcgcaagagaaatgcgtcccatttctcgtcggactcggggaaggaagcgtgatgctctcggtcaagcacgtcgctcgccagcgctgcgaggagttcggccttcgtgcggaagtgccagtagaggccgggctgctgtacctgtaagtgagccgccagcgcgcgagtggtgaagccatcgagcccagtctcgtcgagcacctgccgggccccgagcaacacggacgtgcggtcgagacgcttccggtggtgagtcatagttgcactttatcatcgataactttatcttagataaagtgactgctcgctactctcatctgactgctcgctactctcatcgtggaatcctgacagccgtgctcatcacggcgaccctcgatgctgcagggctgggcctcgtgatgccgatcttgcctacccttctcgaccaggtcggtgcccccgacgacatgatcccactgcacgtcggactactgacagcgctctatgcgatcatgcagtttctttgcgccccgatccttggccgactctctgaccgtttcggacgccgccgcgtgcttgtcgcctccctcgcaggcgcgacgatcgactacctcgtgctcgcactgacggacacgctgtgggtcttttacctcgcccgcgcggttgcaggcattaccggcgccacgaacgccgtcaccgcgacggtgatcgccgacattactccgccggatcagcgcgcaaaacgctacgggtggctcggcgcatgctacggcggtggcatgatcgcgggtcccgccattggcggtcttttcggcggggtctcaccgcatctgccattcctcgtcgccgccgcgctcgccggaatcaccctcgtactcagcgcgagtcttctgcgtgagacgcggccaccgggcagcaacggctcgcacgcacagcaacccggtacggcgaagcgaaccgcagtgccggggatgcttatccttctcgcagtcttcggcatcgtgcagttcatcggccaagcaccaggctccacctgggtgctcttcacgcagcagcgcctcgactggaaccccgtcgaagtcggcgtttcgctatccatcttcggaatggtgcaagtattcgtgcaggcggcactgaccggacgcatcgtgtcccggatcggcgagacccgggcgatcctcgtcggtatcgccgcagacgccattgggctcatcggccttgccctcatcgccagcacatgggcgatgctaccgatcctcgcagcgctcggactcggcagcatcacgttgcccgcactgcagacgctgctctcgagacgcgcgcccgagcagcagcagggacgcctgcagggaacacttgcaagcctgaacagcctcacctcgatcatcggcccggtcaccttcaccggcattttcgcactcacccgaacgaatgcagacggcaccctctggatctgcgccgcagcgctctacgttctctgcgccctcctgatgatccgtgagacatgcgcctcacggcgatctcgataaccgcgctaaggtgccatcccgatgcgacgggatcgctctgccaccagtcaagtctcccgtagccggtatgagggccagcctcgcagagcaggattcccgttgagcaccgccaggtgcgaataagggacagtgaagaaggaacacccgctcgcgggtgggcctacttcacctatcctgccccgctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagccatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattac

SEQ ID NO: 8 - DNA, 29 bp, Artificial Sequence
  Description of Artificial Sequence Synthetic primer
cgggatcctcaggctcgcacgttggaggg

SEQ ID NO: 9 - DNA, 42 bp, Artificial Sequence
  Description of Artificial Sequence Synthetic primer
cccatccactaaacttaaacaaaacgtgaaagaatcataatt

SEQ ID NO: 10 - DNA, 42 bp, Artificial Sequence
  Description of Artificial Sequence Synthetic primer
tgtttaagtttagtggatggggagtgggaggaaatccgcgtt

SEQ ID NO: 11 - DNA, 30 bp, Artificial Sequence
  Description of Artificial Sequence Synthetic primer
gtggatccgcccaaaacaccacggtggcgt

SEQ ID NO: 12 - DNA, 29 bp, Artificial Sequence
  Description of Artificial Sequence Synthetic primer
gaggatccaatcattgctgagctgcgcag

SEQ ID NO: 13 - DNA, 42 bp, Artificial Sequence
  Description of Artificial Sequence Synthetic primer
cccatccactaaacttaaacaatcaacaactgtgggtgttga

SEQ ID NO: 14 - DNA, 42 bp, Artificial Sequence
  Description of Artificial Sequence Synthetic primer
tgtttaagtttagtggatgggtcggtgaagccctggaatgaa

SEQ ID NO: 15 - DNA, 29 bp, Artificial Sequence
  Description of Artificial Sequence Synthetic primer
agggatccgtgatgagtccgatctcggaa

SEQ ID NO: 16 - DNA, 24 bp, Artificial Sequence
  Description of Artificial Sequence Synthetic primer
tggctcacttcgctggtcttgttg

SEQ ID NO: 17 - DNA, 21 bp, Artificial Sequence
  Description of Artificial Sequence Synthetic primer
gaatggttgcggcgagtgtca

SEQ ID NO: 18 - DNA, 23 bp, Artificial Sequence
  Description of Artificial Sequence Synthetic primer
gttgggggagcaggacgagtggt

SEQ ID NO: 19 - DNA, 24 bp, Artificial Sequence
  Description of Artificial Sequence Synthetic primer
tacgcgcatctggaagcctggtta

SEQ ID NO: 20 - DNA, 24 bp, Artificial Sequence
  Description of Artificial Sequence Synthetic primer
cgttccggcatcattctggctaag

SEQ ID NO: 21 - DNA, 24 bp, Artificial Sequence
  Description of Artificial Sequence Synthetic primer
gcgactccgggttgttcctgataa